(19) United States
(12) Patent Application Publication
(10) Pub. No.: US 2026/0144681 A1
(43) Pub. Date: May 28, 2026

(54) EAR WASHING DEVICE WITH WASTE LIQUID WITHDRAWAL FUNCTION

(71) Applicant: Guangdong Okay Technology Co., Ltd., Zhongshan (CN)

(72) Inventor: Rangming ZHANG, Zhongshan (CN)

(73) Assignee: Guangdong Okay Technology Co., Ltd., Zhongshan (CN)

(21) Appl. No.: 18/960,009

(22) Filed: Nov. 26, 2024

(51) Int. Cl. A61F 11/00; A61M 1/00; A61M 3/02

(52) U.S. Cl. CPC A61F 11/006; A61M 1/77; A61M 3/0283

(57) ABSTRACT

An ear washing device with a waste liquid withdrawal function is disclosed, including: a body; an earmuff disposed on the body, the earmuff being provided with a liquid return port at a lower part thereof; a water spray assembly disposed on the body and including a water storage chamber, a first water pump and a sprayer, an input end of the first water pump being communicated with the water storage chamber, an output end of the first water pump being communicated with the sprayer, and the sprayer extending inside the earmuff; and a second water pump, disposed on the body, an input end of the second water pump being communicated with the liquid return port, the body being provided with a waste liquid chamber, and an output end of the second water pump being connected to the waste liquid chamber.

### TECHNICAL FIELD

The present disclosure relates to an ear washing device with a waste liquid withdrawal function.

### BACKGROUND

Ear washing devices are devices that use water to clean the ear canal. A typical ear washing device includes a water spray device and a separate waste liquid box. When in use, the user takes the water spray device with one hand to spray water into the ear canal, and puts the waste liquid box under the ear with the other hand to hold wastewater flowing back from the ear canal. This method requires the coordination of both hands and is troublesome to operate.

### SUMMARY

The present disclosure aims to solve at least one of the technical problems in the existing technology. To this end, an ear washing device is proposed herein, which can be used with one hand and is easy to operate.

An ear washing device with a waste liquid withdrawal function according to an embodiment of a first aspect of the present disclosure includes: a body; an earmuff disposed on the body, the earmuff being provided with a liquid return port at a lower part thereof; a water spray assembly disposed on the body and including a water storage chamber, a first water pump and a sprayer, an input end of the first water pump being communicated with the water storage chamber, an output end of the first water pump being communicated with the sprayer, and the sprayer extending inside the earmuff; and a second water pump, disposed on the body, an input end of the second water pump being communicated with the liquid return port, the body being provided with a waste liquid chamber, and an output end of the second water pump being connected to the waste liquid chamber.

The ear washing device with a waste liquid withdrawal function according to an embodiment of the present disclosure has at least the following beneficial effects.

The earmuff is used to cover an ear, and the sprayer extends inside the earmuff. The sprayer sprays water into the ear canal to clean the ear canal, and the water returned from the ear canal gathers in the earmuff and is pumped away from the liquid return port by the second water pump. In this way, the user merely needs to hold the ear washing device with one hand, which is simple to operate and easy to use.

In some embodiments of the present disclosure, the earmuff is detachably connected to the body. The earmuff is provided with a first through hole, and the sprayer is provided with a first connecting portion that matches the first through hole. When the earmuff is mounted on the body, the first connecting portion is arranged to pass through the first through hole and shield the first through hole.

In some embodiments of the present disclosure, a first magnetic component is disposed on the body, and a second magnetic component is disposed on the earmuff. When the earmuff is mounted on the body, the first magnetic component and the second magnetic component are attracted together.

In some embodiments of the present disclosure, the body is connected with a silicone sleeve having a first accommodation chamber and a second accommodation chamber. The first water pump and the second water pump are respectively provided in the first accommodation chamber and the second accommodation chamber.

In some embodiments of the present disclosure, the silicone sleeve is provided with a first connecting tube and a second connecting tube. The first connecting tube communicates the input end of the first water pump with the water storage chamber, and the second connecting tube communicates the output end of the second water pump with the waste liquid chamber. The first connecting tube, the second connecting tube and the silicone sleeve are of an integrated structure.

In some embodiments of the present disclosure, a battery assembly that powers the first water pump and the second water pump is further included.

In some embodiments of the present disclosure, the waste liquid chamber is provided with a drain outlet. A cover plate is rotatably arranged on the body. The cover plate is provided with a plug portion facing the drain outlet. The cover plate is capable of rotating relative to the body until the plug portion seals the drain outlet.

In some embodiments of the present disclosure, a rotating shaft is disposed on the cover plate, and a lug is disposed on the body. The lug is provided with a shaft hole having a notch, and the rotating shaft is mounted into the shaft hole from the notch.

In some embodiments of the present disclosure, an inner peripheral wall of the earmuff has a flange that extends in a circle.

In some embodiments of the present disclosure, the sprayer includes an extension tube fixed on the body and a nozzle detachably connected to the extension tube.

Additional aspects and advantages of the utility model will be set forth in part in the description which follows, and in part will be obvious from the description, or may be learned by practice of the utility model.

### BRIEF DESCRIPTION OF DRAWINGS

The above and/or additional aspects and advantages of the present disclosure will become apparent and readily understood from the description of the embodiments in conjunction with the following accompanying drawings, in which:

FIG. 1 is a schematic structural view of an ear washing device with a waste liquid withdrawal function according to an embodiment of the present disclosure;

FIG. 2 is a schematic exploded view of the device of FIG. 1 according to an embodiment of the present disclosure;

FIG. 3 is a schematic exploded view of the device of FIG. 1 according to an embodiment of the present disclosure from another perspective;

FIG. 4 is a schematic structural view of an earmuff in FIG. 1; and

FIG. 5 is a schematic structural view of a water tank in FIG. 1.

### REFERENCE NUMERALS

body 100, base 110, first conduit 111, second conduit 112, housing 120, water tank 130, water storage chamber 131, waste liquid chamber 132, drain outlet 1321, lug 133, partition plate 134, first magnetic component 140,
earmuff 200, liquid return port 210, first through hole 220, second magnetic component 230, soft rubber portion 240, flange 241,
sprayer 300, extension tube 310, nozzle 320, first connecting portion 330,
first water pump 410, second water pump 420,
silicone sleeve 510, first accommodation chamber 511, second accommodation chamber 512, first connecting tube 513, the second connecting tube 514, battery assembly 520, circuit board 530, cover plate 540, plug portion 541, rotating shaft 542, connecting port 550.

### DETAILED DESCRIPTION

Embodiments of the present disclosure will be described in detail below, and examples of the embodiments are shown in the accompanying drawings, in which throughout the same or similar reference numerals denote the same or similar elements or elements having the same or similar functions. The following embodiments described with reference to the accompanying drawings are exemplary and serve merely to explain the present disclosure, and should not be construed as limiting the present disclosure.

In the description of the present disclosure, it is to be understood that, referring to orientation description, the instructed orientation or positional relationships, for example, upper, lower, front, rear, left, right, etc., are based on the orientation or positional relationships shown in the accompanying drawings, merely for ease of description of the present disclosure and simplification for the description, rather than indicating or implying that the device or element referred to must have a specific orientation and be constructed and operated in a specific orientation, which, therefore, cannot be construed as limiting the present disclosure.

In the description of the present disclosure, if described, first and second are merely for the purpose of distinguishing technical features, and not to be construed as indicating or implying relative importance or implicitly indicating the number of technical features indicated or implicitly indicating the precedence relationship of technical features indicated.

In the description of the present disclosure, unless explicitly defined otherwise, the terms such as providing, installing, connecting should be understood broadly, and those having ordinary skills in the art may reasonably determine the specific meaning of the above terms in the present disclosure combined with the specific content of the technical solution.

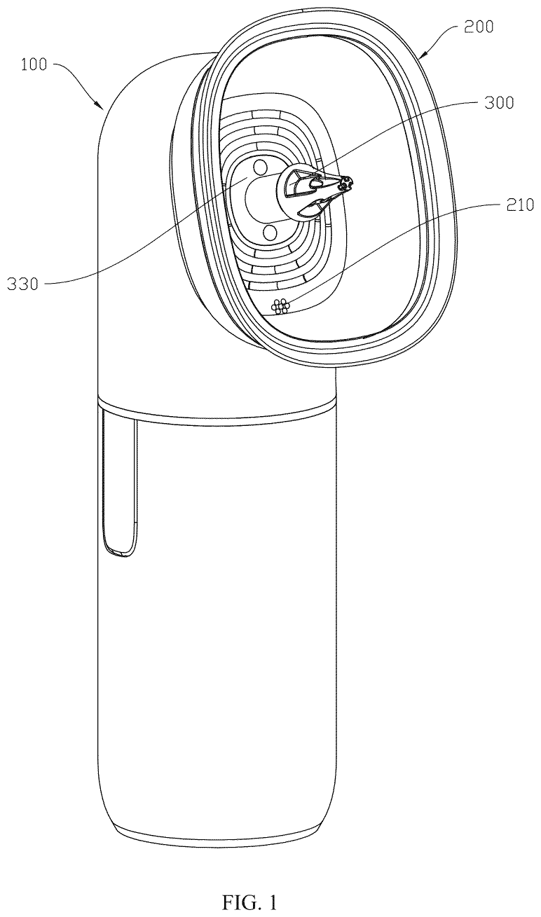

Referring to FIG. 1, an ear washing device with a waste liquid recovery function according to an embodiment of the present disclosure includes a body 100, an earmuff 200, a water spray assembly, and a second water pump 420.

The body 100 includes a base 110 and a housing 120. An installation chamber is defined between the base 110 and the housing 120. A water tank 130 is connected below the base 110.

The earmuff 200 is disposed on the body 100, and the earmuff 200 is provided with a liquid return port 210 at a lower part thereof.

The water spray assembly is disposed on the body 100. The water spray assembly includes a water storage chamber 131, a first water pump 410, and a sprayer 300. An input end of the first water pump 410 is communicated with the water storage chamber 131, and an output end of the first water pump 410 is communicated with the sprayer 300. The sprayer 300 extends inside the earmuff 200.

The second water pump 420 is disposed on the body 100. An input end of the second water pump 420 is communicated with the liquid return port 210. The body 100 is provided with a waste liquid chamber 132. An output end of the second water pump 420 is connected to the waste liquid chamber 132.

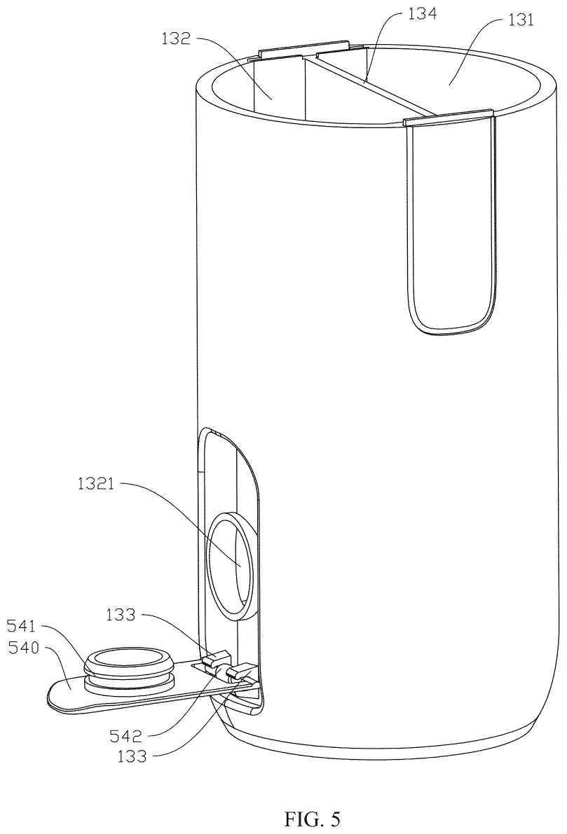

Specifically, as shown in FIG. 5, the water tank 130 has a water storage space inside, and a partition plate 134 is provided in the water storage space. The partition plate 134 divides the water storage space into a water storage chamber 131 and a waste liquid chamber 132. A first conduit 111 and a second conduit 112 are disposed on the bottom plate. The first conduit 111 communicates the water storage chamber 131 with the installation chamber. The first water pump 410 is disposed in the installation chamber, and the input end of the first water pump 410 is communicated with the end of the first conduit 111 away from the water storage chamber 131. The second conduit 112 communicates the waste liquid chamber 132 with the installation chamber. The second water pump 420 is disposed in the installation chamber, and the output end of the second water pump 420 is connected to the end of the second conduit 112 away from the waste liquid chamber 132.

It should be appreciated that the earmuff 200 is used to cover an ear, and the sprayer 300 extends inside the earmuff 200. The sprayer 300 sprays water into the ear canal to clean the ear canal, and the water returned from the ear canal gathers in the earmuff 200 and is pumped away from the liquid return port 210 by the second water pump 420. In this way, the ear washing device is simple to operate and easy to use. In addition, the second water pump 420 absorbs the moisture and gas in the earmuff 200, causing a negative pressure in the earmuff 200. This allows the earmuff 200 to be stably adsorbed on the user's skin.

Referring to FIGS. 1 to 3, in some embodiments of the present disclosure, the earmuff 200 is detachably connected to the body 100. The earmuff 200 is defined with a first through hole 220. The sprayer 300 is provided with a first connecting portion 330 that matches the first through hole 220. When the earmuff 200 is mounted on the body 100, the first connecting portion is arranged to pass through the first through hole and shield the first through hole 220. It should be appreciated that the earmuff 200 may be disassembled for cleaning or replacement. Specifically, the housing 120 is provided with a connecting port 550, and the input end of the second water pump 420 is communicated with the connecting port 550 through a water pipe. When the earmuff 200 is mounted on the body 100, the liquid return port 210 on the earmuff 200 is communicated with the connecting port 550, so that the second water pump 420 can pump the water in the earmuff 200 away from the liquid return port 210.

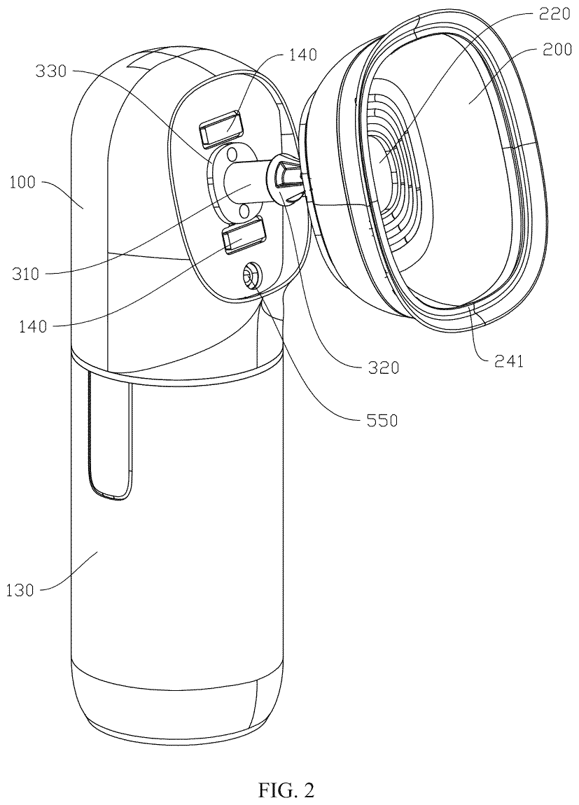

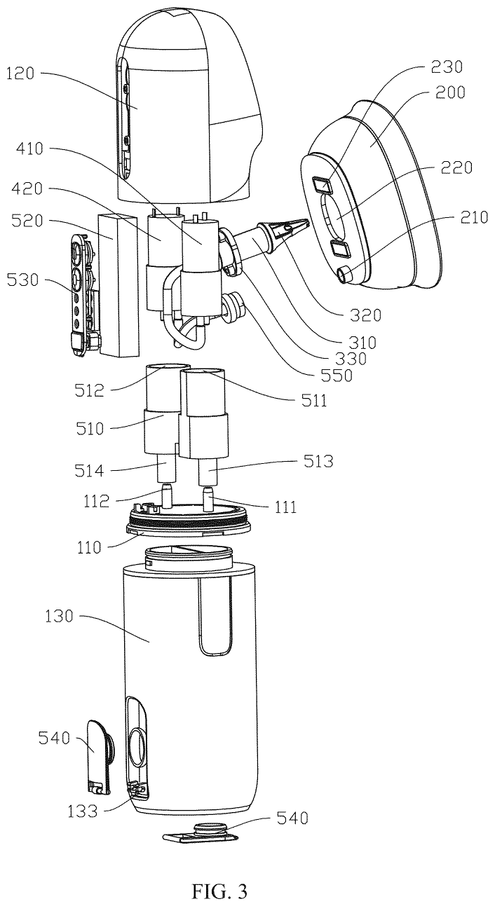

Referring to FIGS. 2 and 3, in some embodiments of the present disclosure, a first magnetic component 140 is disposed on the body 100, and a second magnetic component 230 is disposed on the earmuff 200. When the earmuff 200 is mounted on the body 100, the first magnetic component 140 and the second magnetic component 230 are attracted together. Through the attraction of the first magnetic component 140 and the second magnetic component 230, the earmuff 200 can be quickly installed and removed.

Referring to FIG. 3, in some embodiments of the present disclosure, the body 100 is connected with a silicone sleeve 510 having a first accommodation chamber 511 and a second accommodation chamber 512. The first water pump 410 and the second water pump 420 are respectively provided in the first accommodation chamber 511 and the second accommodation chamber 512. It should be appreciated that the first water pump 410 and the second water pump 420 will generate vibration and noise when working. By arranging the first water pump 410 and the second water pump 420 in the silicone sleeve 510, the silicone sleeve 510 can absorb the vibration of the first water pump 410 and the second water pump 420. In this way, the vibration and noise of the ear washing device during working can be reduced.

Referring to FIGS. 1 and 2, in some embodiments of the present disclosure, the silicone sleeve 510 is provided with a first connecting tube 513 and a second connecting tube 514. The first connecting tube 513 communicates the input end of the first water pump 410 with the water storage chamber 131, and the second connecting tube 514 communicates the output end of the second water pump 420 with the waste liquid chamber 132. The first connecting tube 513, the second connecting tube 514 and the silicone sleeve 510 are of an integrated structure. In this way, no external water pipes are needed for connection, which can reduce the use of water pipes and save costs.

Specifically, the silicone sleeve 510 is disposed in the accommodation chamber, a free end of the first connecting tube 513 is sleeved on the first conduit 111, and a free end of the second connecting tube 514 is sleeved on the second conduit 112. The first connecting tube 513 is communicated with the first accommodating chamber 511. When the first water pump 410 is disposed in the first accommodating chamber 511, the input end of the first water pump 410 extends from the first accommodating chamber 511 into the first connecting tube 513. The second connecting tube 514 is communicated with the second accommodating chamber 512. When the second water pump 420 is extended into the second accommodating chamber 512, the output end of the second water pump 420 extends from the accommodating chamber into the second connecting tube 514. In this way, it is convenient for installation.

Referring to FIG. 3, in some embodiments of the present disclosure, a battery assembly 520 that powers the first water pump 410 and the second water pump 420 is also included. Thus, it is easy to carry. Specifically, the battery assembly 520 is disposed in the installation chamber to better protect the battery assembly 520. A circuit board 530 is also provided in the installation chamber. The circuit board 530 is electrically connected to the battery assembly 520, the first water pump 410 and the second water pump 420, so that the first water pump 410 and the second water pump 420 can be controlled to be turned on or off or their gears can be adjusted.

Referring to FIG. 5, in some embodiments of the present disclosure, the waste liquid chamber 132 is provided with a drain outlet 1321. A cover plate 540 is rotatably arranged on the body 100. The cover plate 540 is provided with a plug portion 541 facing the drain outlet 1321. The cover plate 540 is capable of rotating relative to the body 100 until the plug portion 541 seals the drain outlet 1321. It should be appreciated that the plug portion 541 is disposed on the cover plate 540, and the cover plate 540 is rotatably connected to the body 100 so that the cover plate 540 is always connected to the body 100 to prevent the cover plate 540 and the plug portion 541 from being lost. It should be noted that a water injection hole communicated with the water storage chamber 131 is defined at the bottom of the water tank 130, and another cover plate 540 with the same structure as the cover plate 540 at the drain outlet 1321 is disposed on the body 100 to seal the water injection hole.

Referring to FIG. 5, in some embodiments of the present disclosure, a rotating shaft 542 is disposed on the cover plate 540, and a lug 133 is disposed on the body 100. The lug 133 is provided with a shaft hole having a notch, and the rotating shaft 542 is mounted into the shaft hole from the notch. In this way, the installation and removal of the cover plate 540 is facilitated.

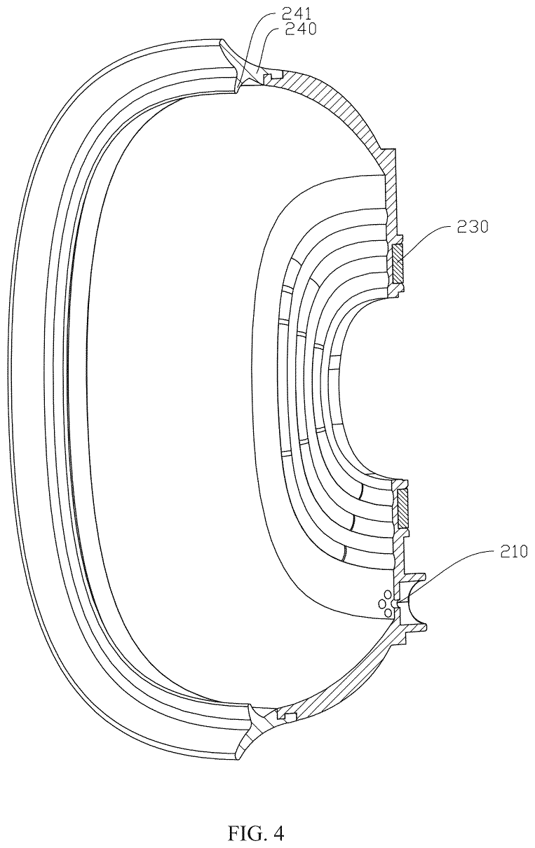

Referring to FIGS. 2 and 4, in some embodiments of the present disclosure, the inner peripheral wall of the earmuff 200 has a flange 241 that extends in a circle. Specifically, the flange 241 is disposed on the inner peripheral surface of the earmuff 200 adjacent to the surrounding edge. The flange 241 can block water flow and prevent water flow from seeping out from the gap between the earmuff 200 and the skin. The body 100 of the earmuff 200 is made of hard plastic, and a soft rubber portion 240 that is easily deformed is connected on the periphery of the earmuff 200. The deformation of the soft rubber portion 240 can better fit the skin and prevent water from flowing out. The flange 241 and the soft rubber portion 240 are of an integrated structure, which facilitates production.

Referring to FIGS. 1 and 2, in some embodiments of the present disclosure, the sprayer 300 includes an extension tube 310 fixed on the body 100 and a nozzle 320 detachably connected to the extension tube 310. In this way, the nozzle 320 is conveniently cleaned and replaced. Specifically, the extension tube 310 is made of hard plastic, and thus can be stably fixed on the body 100. The sprayer 300 is made of soft material such as rubber, which is beneficial to protecting the user's skin.

The embodiments of the present disclosure have been described in detail above in conjunction with the accompanying drawings. However, the present disclosure is not limited to the above embodiments. Within the scope of knowledge possessed by those of ordinary skill in the art, various changes may also be made without departing from the purpose of the present disclosure.

What is claimed is:

1. An ear washing device with a waste liquid withdrawal function, comprising:
a body;
an earmuff disposed on the body, the earmuff being provided with a liquid return port at a lower part of the earmuff;
a water spray assembly disposed on the body and comprising a water storage chamber, a first water pump and a sprayer, an input end of the first water pump being communicated with the water storage chamber, an output end of the first water pump being communicated with the sprayer, and the sprayer extending inside the earmuff; and
a second water pump disposed on the body, an input end of the second water pump being communicated with the liquid return port, the body being provided with a waste liquid chamber, and an output end of the second water pump being connected to the waste liquid chamber.

2. The ear washing device with a waste liquid withdrawal function according to claim 1, wherein the earmuff is detachably connected to the body, the earmuff is provided with a first through hole, the sprayer is provided with a first connecting portion that matches the first through hole, and when the earmuff is mounted on the body, the first connecting portion is arranged to pass through the first through hole and shield the first through hole.

3. The ear washing device with a waste liquid withdrawal function according to claim 2, wherein a first magnetic component is disposed on the body, a second magnetic component is disposed on the earmuff, and when the earmuff is mounted on the body, the first magnetic component and the second magnetic component are attracted together.

4. The ear washing device with a waste liquid withdrawal function according to claim 1, wherein the body is connected with a silicone sleeve having a first accommodation chamber and a second accommodation chamber, and the first water pump and the second water pump are provided in the first accommodation chamber and the second accommodation chamber, respectively.

5. The ear washing device with a waste liquid withdrawal function according to claim 4, wherein the silicone sleeve is provided with a first connecting tube and a second connecting tube, the first connecting tube communicates the input end of the first water pump with the water storage chamber, the second connecting tube communicates the output end of the second water pump with the waste liquid chamber, and the first connecting tube, the second connecting tube and the silicone sleeve are of an integrated structure.

6. The ear washing device with a waste liquid withdrawal function according to claim 1, further comprising a battery assembly for powering the first water pump and the second water pump.

7. The ear washing device with a waste liquid withdrawal function according to claim 4, wherein the waste liquid chamber is provided with a drain outlet, a cover plate is rotatably arranged on the body, the cover plate is provided with a plug portion facing the drain outlet, and the cover plate is capable of rotating relative to the body until the plug portion seals the drain outlet.

8. The ear washing device with a waste liquid withdrawal function according to claim 7, wherein a rotating shaft is disposed on the cover plate, a lug is disposed on the body, the lug is provided with a shaft hole having a notch, and the rotating shaft is mounted into the shaft hole from the notch.

9. The ear washing device with a waste liquid withdrawal function according to claim 5, wherein an inner peripheral wall of the earmuff has a flange that extends in a circle.

10. The ear washing device with a waste liquid withdrawal function according to claim 5, wherein the sprayer comprises an extension tube fixed on the body and a nozzle detachably connected to the extension tube.
